(19) United States
(12) Patent Application Publication
(10) Pub. No.: US 2024/0200080 A1
(43) Pub. Date: Jun. 20, 2024

(54) DIAGNOSTIC DEVICE FOR LOCAL INTRAVENOUS BIOMARKER MEASUREMENTS AND METHODS OF USE THEREOF

(71) Applicant: Cook Medical Technologies LLC, Bloomington, IN (US)

(72) Inventors: Grainne Leonard, Co. Dublin (IE); Jennifer O'Malley, Co. Limerick (IE); Diarmuid McGee, Vancouver BC (CA); Criostoir O Bhealtun, Co Clare (IE); David Slattery, Limerick (IE); Kevin Chmura, Winston-Salem, NC (US)

(73) Assignee: Cook Medical Technologies LLC, Bloomington, IN (US)

(21) Appl. No.: 18/540,229

(22) Filed: Dec. 14, 2023

Related U.S. Application Data
(60) Provisional application No. 63/387,781, filed on Dec. 16, 2022.

(51) Int. Cl. C12N 15/113; A61B 5/0215; A61B 5/15; A61P 17/06; A61P 37/06; C07K 14/54; C07K 14/65; C12N 9/16

(52) U.S. Cl. CPC C12N 15/1138; A61B 5/0215; A61B 5/15003; A61B 5/150412; A61P 17/06; A61P 37/06; C07K 14/5406; C07K 14/65; C12N 9/16; C12N 2310/14; C12Y 301/03001

(57) ABSTRACT

A device including a needle configured to measure portal pressure in a vein of a mammal and provide an indication of a measurement a diagnostic biomarker in a sample drawn from the vein before removing the needle is provided. Methods of providing medical data about a patient by use of a device are further provided.

### CROSS-REFERENCE TO RELATED APPLICATIONS

This application is a non-provisional application which claims priority under 35 USC § 119 to U.S. provisional application Ser. No. 63/387,781, filed Dec. 16, 2022, which is incorporated by reference herein in its entirety.

### FIELD

The present disclosure relates to methods for use of medical devices. More particularly, the present disclosure relates to methods for measuring diagnostic biomarkers intravenously.

### BACKGROUND

The statements in this section merely provide background information related to the present disclosure and may constitute prior art.

The portal vein is a blood vessel that moves blood from the spleen and gastrointestinal tract to the liver's capillary beds. The hepatic veins are the carriers of deoxygenated blood that flows via the liver, and drain the blood into the inferior vena cava. The hepatic veins are also the carriers of blood that has been drained from the colon, pancreas, small intestine, and stomach, and cleaned by the liver. The hepatic veins originate from the core vein of the liver lobule. The deoxygenated blood from the hepatic veins into the inferior vena cava is delivered back to the heart, wherein the re-oxygenation process of the blood begins.

Abnormally high blood pressure in the portal vein is known as “portal hypertension.” Portal hypertension is a major—and the most common—complication of liver cirrhosis, and the leading cause of mortality associated with liver cirrhosis. Further, cirrhosis is the leading cause of portal hypertension. Portal hypertension may cause the growth of collateral blood vessels that bypass the liver, resulting in the circulation of unprocessed substances throughout the body. Portal pressure falling within a range of about 0 to about 5 mmHg is normal; portal pressure falling within a range of about 6 to about 9 mmHg may be considered “borderline”; portal pressure of about 10 mmHg may be considered hypertensive; and when portal pressure exceeds about 12 mmHg, portal hypertension is present and varices may be at risk of rupture.

An increase in portal hypertension may lead to the development of portosystemic collateral vessels and a hyperkinetic circulation, which often results in serious medical consequences such as varices, ascites, bacterial peritonitis, hepatorenal syndrome, hepatic encephalopathy, enteropathy, colopathy, and abnormal liver metabolism. According to the American Association for the Study of Liver Diseases and the American College of Gastroenterology in an updated practice guideline from 2007, the preferred method for assessing portal pressure is the Hepatic Venous Pressure Gradient (“HPVG”) measurement, which is obtained by placing a catheter in the hepatic vein and wedging the catheter into a small branch, or by inflating a balloon and occluding a larger branch of the hepatic vein. The European Association for the Study of the Liver recommends both HVPG and upper gastrointestinal endoscopy as the standard method for quantifying portal hypertension. Additional methods for diagnosis of portal hypertension may include, but are not limited to, transient elastography, magnetic resonance elastography, point shear wave elastography, acoustic radiation force impulse imaging, and serum biomarkers.

### SUMMARY

In an example, the present disclosure provides a medical device. The medical device includes a tubular body defining a lumen extending longitudinally therethrough, the tubular body defining a longitudinal axis. The tubular body includes a needle including the lumen extending longitudinally therethrough. The tubular body includes a handle proximal to the needle. The tubular body further includes a stopcock proximal to the handle, the stopcock configured to close or open the lumen to flow of a sample proximal to the stopcock. The tubular body further includes a connecting tube proximal to the stopcock, the connecting tube including the lumen. The tubular body further includes a pressure transducer proximal to the connecting tube. The tubular body further includes a syringe proximal to the pressure transducer. The medical device further includes a sample measurement device attached to the handle or to the needle near a distal tip of the needle. The device is configured to provide a measurement of portal pressure in a vein of a mammal and an indication of a measurement of a diagnostic biomarker in a sample from the vein before the needle is removed from the vein. The sample measurement device may be configured to detect a bilirubin concentration. The sample measurement device may be a digital microfluidic biochip. The diagnostic biomarker may include: a white blood cell count, a red blood cell count, a platelet count, a hematocrit, one or more of the red blood cell indices, or combinations thereof. The diagnostic biomarker may include apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, or combinations thereof. The diagnostic biomarker may include circulating tumor cells, exosomes, or combinations thereof. The diagnostic biomarker may include Alpha FetoProtein. The mammal may be a human. The vein may be the hepatic vein, the portal vein, or both. The sample measurement device may be configured to communicate an indication of a measurement to an external receiver.

In another example, the present disclosure provides a method of providing medical data about a human. The method includes: inserting a needle into a vein of the mammal; measuring the portal pressure in the vein; collecting a sample from the vein; measuring a diagnostic biomarker within the sample; and removing the needle from the vein; wherein an indication of a measurement of the diagnostic biomarker is provided prior to the removing. The device may further include a digital microfluidic biochip. The diagnostic biomarker may include a white blood cell count, a red blood cell count, a platelet count, a hematocrit, one or more of the red blood cell indices, or combinations thereof. The diagnostic biomarker may include apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, or combinations thereof. The diagnostic biomarker may include circulating tumor cells, exosomes, or combinations thereof. The device may include a test cassette connected to the needle. The diagnostic biomarker may include Alpha FetoProtein. The measuring the diagnostic biomarker may include: providing an amount of the sample from the needle to the test cassette; adding buffer to the sample in the test cassette; transmitting a current to the sample in the test cassette; and reading a test result on the test cassette. The vein may be the hepatic vein, the portal vein, or both. The device may further include a pressure transducer. The device may further include a sample measurement device attached to the needle near a distal tip of the needle, the sample measurement device configured to communicate a measurement to an external receiver. The diagnostic biomarker may be bilirubin. The mammal may be a human.

In yet another example, the present disclosure provides a method of providing medical data about a mammal. The method includes: inserting a needle of a device into a vein of the mammal, the device further including a digital microfluidic biochip; measuring the portal pressure in the vein; collecting a sample from the vein; measuring a diagnostic biomarker within the sample; and removing the device from the vein. An indication of a measurement of the diagnostic biomarker is provided prior to the removing. The diagnostic biomarker includes: a white blood cell count, a red blood cell count, a platelet count, a hematocrit, one or more of the red blood cell indices, apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, circulating tumor cells, exosomes, or combinations thereof.

Further areas of applicability will become apparent from the description provided herein. It should be understood that the description and specific examples are intended for purposes of illustration only and are not intended to limit the scope of the present disclosure.

### DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts through the different views.

FIG. 1 illustrates a perspective view of an example of a device configured to draw a sample from a vein of a mammal according to the principles of the present disclosure;

FIG. 2 illustrates a perspective view of an example of a digital microfluidic biochip according to the principles of the present disclosure;

FIG. 3 illustrates a partial top view of an example of a two-dimensional microfluidic array according to the principles of the present disclosure;

FIG. 4 illustrates a side view of an example of a two-dimensional microfluidic array according to the principles of the present disclosure;

FIGS. 5A, 5B, 50, 5D, 5E, 5F, and 5G illustrate examples of test cassettes demonstrating possible biomarker diagnostic test results that may be obtained according to the principles of the present disclosure;

FIG. 6A illustrates an example of a portal pressure gradient measurement device taking a sample of blood from a patient's hepatic vein; and

FIG. 6B illustrates an example of a portal pressure gradient measurement device taking a sample of blood from a patient's portal vein.

The drawings described herein are for illustration purposes only and are not intended to limit the scope of the present disclosure in any way.

### DETAILED DESCRIPTION

The following description presents examples and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axis that aligns with the proximal-distal axis of the device (or component).

The uses of the terms “a” and “an” and “the” and similar referents in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of,” the examples or elements presented herein, whether explicitly set forth or not.

In describing elements of the present disclosure, the terms 1^{st}, 2^{nd}, first, second, A, B, (a), (b), and the like may be used herein. These terms are only used to distinguish one element from another element, but do not limit the corresponding elements irrespective of the nature or order of the corresponding elements.

Unless otherwise stated, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of +15%, or less, of the numerical value. For example, a value differing by +15%, +14%, +10%, or +5%, among others, would satisfy the definition of “about,” unless more narrowly defined in particular instances.

As used herein, the term “sample” refers to one or more cells, whole blood, body fluid, any cell-containing blood fraction, a fragmented tumor, a tumor cell suspension, or a cell culture established from a patient's sample.

As used herein, the term “biomarker” refers to a measurable indicator of a biological state or condition. For example, a biomarker may be a substance, the detection of which indicates a particular disease state, or indicates a change in expression or state of a protein that correlates with the risk or progression of a disease or with the susceptibility of the disease to a given treatment.

As used herein, the term nonalcoholic fatty liver disease (“NAFLD”) refers to a disease characterized by excessive fat buildup in the liver without another clear cause, such as alcohol use. Some individuals diagnosed with NAFLD may develop nonalcoholic steatohepatitis (“NASH”), which is an aggressive form of fatty liver disease characterized by liver inflammation that may progress to advanced scarring (cirrhosis) and liver failure, similar to the damage caused by heavy alcohol use. Circulating serum biomarkers of liver fibrosis may give moderate estimates in the diagnosis of liver fibrosis and cirrhosis, including, for example, without limitation, the ratio of the transaminase liver enzyme aspartate aminotransferase (“AST”) to platelets in the blood (known as the AST/platelet ratio index, or the “APRI” score), Fibrotest, FIB-4, and the NAFLD fibrosis score. Examples of biomarkers of NAFLD may include apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, and leucine-rich alpha-2-glycoprotein.

Alpha-1-acid glycoprotein 2 and leucine-rich alpha-2-glycoprotein may each demonstrate decreasing levels of protein with increasing NAFLD severity. Levels of cytostatin-C, lipopolysaccharide-binding protein, and IgG Fc-binding protein may each demonstrate increasing levels with increasing NAFLD severity.

Apolipoprotein F may demonstrate a decrease in concentration with increasing NAFLD severity. Accordingly, monitoring changes in the level of apolipoprotein F in an individual provides an indication of the progress of NAFLD through the stages of severity of NAFLD.

Apolipoprotein D may demonstrate high levels in healthy controls, and consistently lower levels in all stages of NAFLD. Accordingly, apolipoprotein D may be used as an early NAFLD biomarker. A decreasing level of apolipoprotein D, as compared with a control sample or reference sample/level, may indicate increasing severity of NAFLD.

Apolipoprotein M may demonstrate a slight decrease from steatosis to NASH stage FO, but reduce an expression in NASH stage F3. Accordingly, apolipoprotein M may be useful in determining progression toward hepatic fibrosis.

Kininogen-1 demonstrates a consistent decrease across all stages of NAFLD.

Ficolin-2 demonstrates a decrease in NAFLD stages, with a lowest level in fibrosis stage F3.

Thrombospondin-1 demonstrates an increase from control levels to steatosis and across all stages of NAFLD.

As used herein, the term “complete blood count” (“CBC”) refers to a set of medical tests that provide information about the cells in a mammal's blood. The CBC indicates the counts of white blood cells, red blood cells, and platelets, the concentration of hemoglobin, and the volume percentage of red blood cells (“hematocrit”). Further, the CBC includes the red blood cell indices and a white blood cell differential. The hematocrit is normally from about 40.7% to about 50.3% for males and from about 36.1% to about 44.3% for females.

As used herein, the term “red blood cell indices” refers to blood tests that may provide information about hemoglobin content and size of red blood cells. Examples of red blood cells indices may include: mean corpuscular volume (or mean cell volume, or “MCV”); mean corpuscular hemoglobin (“MCH”); and mean corpuscular hemoglobin concentration (“MCHC”).

As used herein, the term “bilirubin” (“BR”) refers to a red-orange compound occurring in the normal catabolic pathway that breaks down heme in vertebrates, which is a necessary process in the mammal's clearance of waste products from the destruction of aged or abnormal red blood cells. A healthy liver should remove bilirubin from the blood. By measuring the gradient in the total serum bilirubin level in blood in the portal vein, approaching the liver, compared to the total serum bilirubin level in blood in the hepatic vein, departing the liver, an indication of liver function may be provided. Elevated levels of bilirubin may indicate poor liver function. The total serum bilirubin level measures both conjugated bilirubin and unconjugated bilirubin. For example, a normal value for a bilirubin test may be about 1.2 milligrams per deciliter (mg/dL) of total bilirubin for adult humans, and may be about 1 mg/dl for humans less than 18 years old. Bilirubin colorimetric assay utilizes the Jendrassik-Grof principle to detect bilirubin, by which total bilirubin concentration may be determined in the presence of a diazo-salt catalyst by formation of azobilirubin, which absorbs at 600 nm, and in the absence of catalyst, which absorbs at 550 nm.

As used herein, the term “mean corpuscular volume” (MCV) refers to a measure of the average volume of a red blood cell. The MCV may be obtained according to formula (1) below, which illustrates that the MCV is obtained by dividing the hematocrit (“Hct”) by the concentration of red blood cells (“[RBC]”):

$$MCV = \frac{H c t}{[ R B C ]} . \qquad ( 1 )$$

MCV is typically expressed in femtoliters (fL, or 10⁻¹⁵ L), and [RBC] in millions per microliter (10⁶/μL). The normal range for MCV for humans may be from about 80 to about 100 fL. In a patient with anemia, the MCV measurement may allow classification of the patient as a microcytic anemia (MCV below normal range), normocytic anemia (MCV within normal range), or macrocytic anemia (MCV above normal range).

As used herein, the term “mean corpuscular hemoglobin” (MCH) refers to a measure of the average mass of hemoglobin (“Hb”) per red blood cell in a sample of blood. The MCH may be obtained according to formula (2) below, which illustrates that the MCH is obtained by dividing the total mass of hemoglobin by the number of red blood cells in a volume of blood:

$$MCH = \frac{H b}{R B C} . \qquad ( 2 )$$

MCH is typically expressed in picograms (pg)/cell. The normal range for MCH for humans may be from about 27 to about 31 (pg)/cell. The MCH may depend on hemoglobin synthesis and the size of the RBC. The MCH may decrease when Hb synthesis is reduced, or when red blood cells are smaller than normal, such as in iron deficiency anemia.

As used herein, the term “mean corpuscular hemoglobin concentration” (MCHC) refers to a measure of the concentration of hemoglobin in a given volume of packed red blood cell. The MCHC may be obtained according to formula (3) below, which illustrates that the MCHC may be obtained by dividing the hemoglobin by the hematocrit:

$$MCHC = \frac{H b}{H c t} . \qquad ( 3 )$$

MCHC may be expressed in g/dL, with a normal range for humans of from about 32 to 36 g/dL. Alternatively, the MCHC may be obtained according to formula (4) below, which illustrates that the MCHC may be measured in percentage (%), as if the MCHC were a mass fraction:

$$MCHC = \frac{{m}_{H b}}{{m}_{R B C}} . \qquad ( 4 )$$

Numerically, the MCHC measured in g/dL and the mass fraction of hemoglobin in red blood cells measured in % may be identical.

As used herein, the term “red blood cell count” refers to the number of red blood cells per volume of blood. For example, a normal range of red blood cell count values in adult humans is generally considered to be from about 4.35 to about 5.65 million red blood cells per microliter (“mcL”) of blood for men, and from about 3.92 to about 5.13 million red blood cells per mcL of blood for women.

As used herein, the term “white blood cell count” refers to the number of white blood cells per volume of blood. For example, a normal range of white blood cell count in adult humans is generally considered to be from about 4,500 to about 11,000 white blood cells per microliter (mcL) of blood.

As used herein, the term “platelet concentration” refers to the number of platelets per unit of volume. For example, a normal range for platelet concentration in adult humans is generally considered to be from about 150,000 to about 500,000 per microliter of blood.

As used herein, the term “white blood cell differential” refers to a test that measures the amounts of the normal white blood cell types, including neutrophils, lymphocytes, monocytes, eosinophils, and basophils, and any abnormal cell types present in a sample. For example, a normal range for neutrophils in adult humans is generally considered to be from about 1,700 to about 7,500 per microliter of blood. For example, a normal range for lymphocytes in adult humans is generally considered to be from about 1,000 to about 3,200 per microliter of blood. For example, a normal range for monocytes in adult humans is generally considered to be from about 100 to about 1,300 per microliter of blood. For example, a normal range for eosinophils in adult humans is generally considered to be from less than about 100 to about 300 per microliter of blood. For example, a normal range for basophils in adult humans is generally considered to be from less than about 100 to about 200 per microliter of blood.

As used herein, the term “Alpha FetoProtein” (“AFP”) refers to a glycoprotein produced in the liver and found in the blood plasma. For example, healthy adult humans should have a low level of AFP in the body of from about 10 ng/ml to about 20 ng/mL. A level of AFP of greater than 200 ng/mL from venous blood may indicate hepatic cirrhosis. A level of AFP of greater than 400 ng/ml may indicate a presence of liver tumors, and reduced levels of AFP may indicate if liver tumors are reducing in size and/or if treatment is successful. AFP plasma level requires a specific blood test.

As used herein, the term “circulating tumor cells” (“CTCs”) refers to tumor cells that diffuse into the circulating blood, and may serve an important role in the progress of cancer. During the early stages of cancer, CTCs may undergo an epithelial-mesenchymal transition and obtain a more invasive phenotype. Subsequently, the CTCs may enter the circulating blood with the aid of immune cells, and enter a dormant state upon reaching distal organs. As a tumor progresses, metastasis may occur under certain conditions. The capture technologies available for CTCs may be based on antibody-based capture, or capture based on the physical properties of CTCs, as well as modern technologies that integrate both types of capture. Emerging modern technologies have increased the accuracy and efficiency of tumor cell capture, and have improved the understanding of tumor cells as well as molecular mechanisms underlying the properties of tumor cells. Further, CTCs may serve an important role in disease progression, prediction of patient prognosis, and individualized treatment.

As used herein, the term “exosomes” refers to membrane-bound extracellular vesicles that may be produced in the endosomal compartment of most eukaryotic cells. A multivesicular body (“MVB”) is an endosome with intraluminal vesicles (“ILVs”) that bud inward into the endosomal lumen. If the MVB fuses with the cell surface, the ILVs may be released as exosomes. Exosomes may carry markers of the respective cells of origin, and may have specialized functions in physiological processes, from coagulation and intercellular signaling to waste management. Exosomes may play a crucial role in regulating tumor growth, metastasis, and angiogenesis in the process of cancer development. Exosomes may carry distinct proteo-transcriptomic signatures that are different from their cancer cells of origin; consequently, there is a growing interest in clinical applications of exosomes as biomarkers, prognostic markers, grading bases, and/or therapies. Components within exosomes may include proteins, deoxyribonucleic acid (“DNA”), ribonucleic acid (“RNA”), messenger RNA (“mRNA”), microRNA, long noncoding RNA, and circular RNA.

In an example, the present disclosure provides methods of measuring diagnostic biomarkers intravenously by use of a device configured to draw a sample from a vein of a mammal including a human in addition to measuring portal pressure under under endoscopic ultrasound in the vein.

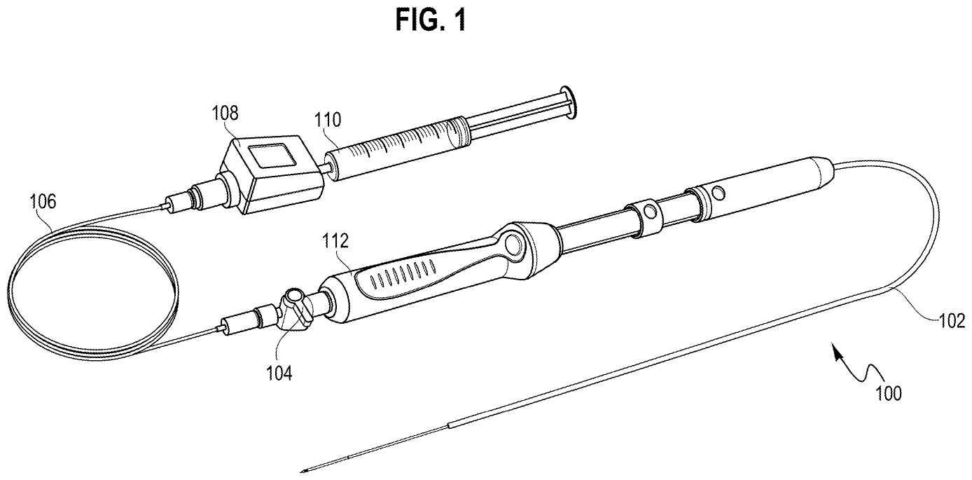

Referring to FIG. 1, a perspective view of an example of a device 100 configured to measure portal pressure in a vein of a mammal under endoscopic ultrasound and to draw a sample from the vein is illustrated. The device 100 may include a tubular body defining a lumen extending longitudinally therethrough, the tubular body defining a longitudinal axis. The distal end of device 100 includes a needle 102 configured to access the hepatic vein and/or the portal vein. Needle 102 includes the lumen extending longitudinally therethrough.

The device 100 includes a needle 102. Needle 102 may be of a size within a range of needle 102 sizes of from 1 French (“Fr.”) to 10 Fr. As will be understood to those of skill in the art, the French scale or French gauge system commonly used to measure the size of a catheter refers to three times the length, measured in millimeters (“mm”) of the outer diameter of the needle 102. For example, a round needle 102 of 1 French will have an outer diameter of ⅓ millimeters; a round needle 102 of 3 French will have an outer diameter of 1 millimeters. In certain examples, needle 102 may have a catheter size of from 1 French to 10 French, including from 1 French, or from 2 French, or from 3 French, or from 4 French, or from 5 French, or from 6 French, or from 7 French, or from 8 French, or from 9 French to 10 French; or to from 1 French to 2 French, or to 3 French, or to 4 French, or to 5 French, or to 6 French, or to 7 French, or to 8 French, or to 9 French, or to 10 French; or any other range of one of the above minima to one of the above maxima. Needle 102 is connected to a distal end of a handle 112. The handle 112 includes the lumen extending longitudinally therethrough through which a sample flows from needle 102 to stopcock 104. A proximal end of handle 112 is fluidly connected to stopcock 104. Stopcock 104 may be configured to control flow of a sample from the mammal proximally into device 100 by manipulating stopcock 104 to close off the lumen from flow proximal to stopcock 104 or open the lumen to flow proximal to stopcock 104. A distal end of connecting tube 106 is fluidly connected to a proximal end of stopcock 104. Connecting tube 106 includes the lumen. A proximal end of connecting tube 106 is fluidly connected to pressure transducer 108.

Pressure transducer 108 is a device that may convert pressure into an analog electrical signal. Without being bound by theory, conversion of pressure into an electrical signal may be achieved by physical deformation of strain gages that are bonded into the diaphragm of pressure transducer 108. Pressure applied to pressure transducer 108 may produce a deflection of the diaphragm that may introduce strain to the gages. The strain may produce an electrical resistance change proportional to the pressure.

In certain examples, pressure transducer 108 may include a sample measurement device (not shown), which may detect a bilirubin concentration. A sample flowing upward through the lumen from needle 102 may be directed into the sample measuring device. In other examples, the sample measurement device is embedded close to the distal tip of needle 102. A sample may be measured by the sample measurement device and the reading may be communicated to an external receiver, which may include a readout. In still other examples, the sample measurement device may be attached to handle 112.

Syringe 110 is fluidly connected to a proximal end of pressure transducer 108 and is configured to draw a sample into needle 102 by pulling the plunger of syringe 110 proximally.

In an example, a method of measuring diagnostic biomarkers intravenously by use of a device 100 configured to draw a sample from a hepatic vein and/or a portal vein of a mammal may include use of a device 100 including a digital microfluidic biochip 200 connected to handle 112 or pressure transducer 108.

In other examples of devices (not shown), examples of a device for acquiring a sample to measure a diagnostic biomarker intravenously may include a biopsy needle.

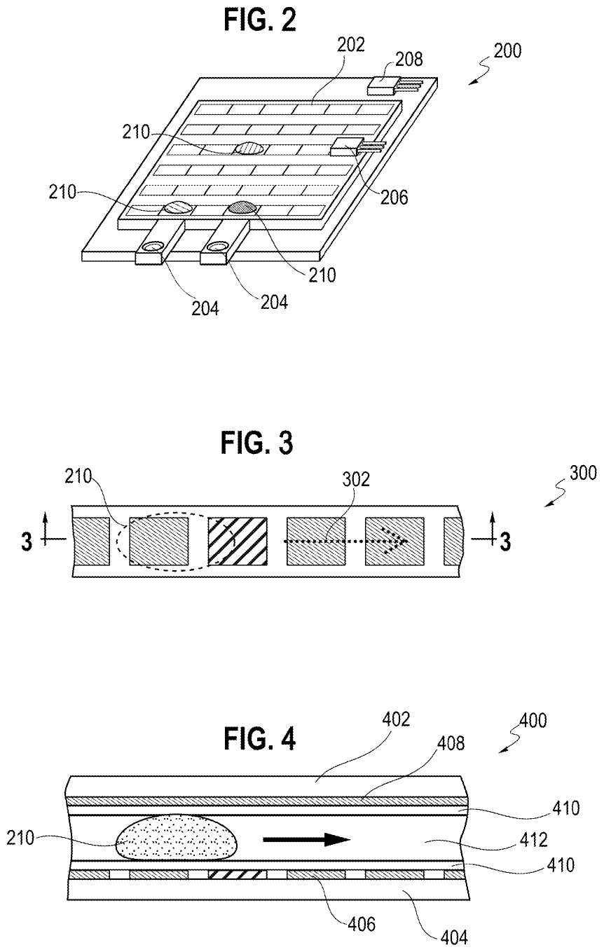

Referring to FIG. 2, a perspective view of an example of a digital microfluidic biochip 200 is illustrated. The digital microfluidic biochip 200 includes a two-dimensional microfluidic array 202, one or more dispensing ports 204, an optical detector 206, and a photodiode 208. The two-dimensional microfluidic array 202 includes a set of basic cells 300, 400 including two parallel glass plates, including top plate 402 and bottom plate 404 illustrated in FIG. 4. The one or more dispensing ports 204 may generate droplets 210 from a sample taken by device 100. The optical detector 206 may detect droplets 210. The photodiode 208 may be configured to send data obtained about a droplet 210 to a receiver (not shown).

Referring to FIG. 3, a partial top view of an example of a basic cell 300 is illustrated. By independently controlling the voltage of electrodes, a droplet 210 may be moved along a line of electrodes by the principle of electrowetting-on-dielectric (“EWOD”), as illustrated by directional arrow 302. By EWOD, many fluidic operations such as mixing and dilution may be performed anywhere in the two-dimensional microfluidic array 202 within different time intervals.

Referring to FIG. 4, a side view of an example of a basic cell 300 is illustrated by cross-section taken along axis 3. Attached to the top surface of bottom plate 404 is a patterned array of individually controllable electrodes 406. The bottom surface of top plate 402 is coated with a continuous ground electrode 408. Coated on the bottom of continuous ground electrode 408 and the top of the patterned array of individually controllable electrodes 406 is hydrophobic insulation 410. Filler medium 412 is sandwiched between top plate 402 and bottom plate 404.

In an example, a method of measuring diagnostic biomarkers intravenously by use of a device 100 including a digital microfluidic biochip 200 may include providing an indication of a value of a complete blood count of a mammal in a droplet 210 of a sample and transmitting the value by photodiode 208 to a receiver. In certain examples, a method may include providing an indication of a value of a white blood cell count in a droplet 210 of a sample. In other examples, a method may include providing an indication of a value of a red blood cell count in a droplet 210 of a sample. In still other examples, a method may include providing an indication of a value of a platelet count in a droplet 210 of a sample. In still other examples, a method may include providing an indication of a value of hematocrit in a droplet 210 of a sample. In still other examples, a method may include providing an indication of a value of one or more of the red blood cell indices in a droplet 210 of a sample. In still other examples, a method may include providing an indication of a value of a white blood cell differential in a droplet 210 of a sample.

In an example, a method of measuring diagnostic biomarkers intravenously by use of a device 100 including a digital microfluidic biochip 200 may include providing an indication of a value of one or more of the red blood indices in a droplet 210 of a sample and transmitting the value by photodiode 208 to a receiver. In certain examples, a method may include providing an indication of a value of mean corpuscle volume in a droplet 210 of a sample. In other examples, a method may include providing an indication of a value of mean corpuscular hemoglobin in a droplet 210 of a sample. In still other examples, a method may include providing an indication of a value of mean corpuscular hemoglobin concentration in a droplet 210 of a sample.

In an example, a method of measuring diagnostic biomarkers intravenously by use of a device 100 including a digital microfluidic biochip 200 may include providing an indication of a value of one or more biomarkers of NAFLD in a droplet 210 of a sample. In certain examples, a method may include providing an indication of a value of apolipoprotein F in a droplet 210 of a sample. In other examples, a method may include providing an indication of a value of lipopolysaccharide-binding protein in a droplet 210 of a sample. In still other examples, a method may include providing an indication of a value of ficolin-2 in a droplet 210 of a sample. In still other examples, a method may include providing an indication of a value of apolipoprotein D in a droplet 210 of a sample. In still other examples, a method may include providing an indication of a value of kininogen-1 in a droplet 210 of a sample. In still other examples, a method may include providing an indication of a value of apolipoprotein M in a droplet 210 of a sample. In still other examples, a method may include providing an indication of a value of thrombospondin-1 in a droplet 210 of a sample. In still other examples, a method may include providing an indication of a value of IgG F C-binding protein in a droplet 210 of a sample. In still other examples, a method may include providing an indication of a value of cystatin-C in a droplet 210 of a sample. In still other examples, a method may include providing an indication of a value of alpha-1-acid glycoprotein 2 in a droplet 210 of a sample. In still other examples, a method may include providing an indication of a value of leucine-rich alpha-2-glycoprotein in a droplet 210 of a sample.

In an example, a method of measuring diagnostic biomarkers intravenously by use of a device 100 including a digital microfluidic biochip 200 may include providing an indication of the presence of circulating tumor cells in a droplet 210 of a sample. In another example, a method may include providing an indication of the presence of exosomes in a droplet 210 of a sample.

Compared to normal blood measurements, methods of the present disclosure may advantageously provide an indication of one or more values of a complete blood cell count from a droplet 210 of a sample within 20 minutes rather than 2-3 days. Further, compared to normal blood measurements, methods of the present disclosure may advantageously provide greater information to a clinician during a procedure performed on a mammal rather than having to wait 2-3 days for the information. Further, compared to normal blood measurements, measurements of the present disclosure may advantageously use lower amounts of reagents. Further, compared to normal blood measurements, measurements of the present disclosure may advantageously require a lower volume of blood. Further, compared to normal blood measurements, measurements of the present disclosure may advantageously reduce the necessity of multiple blood tests.

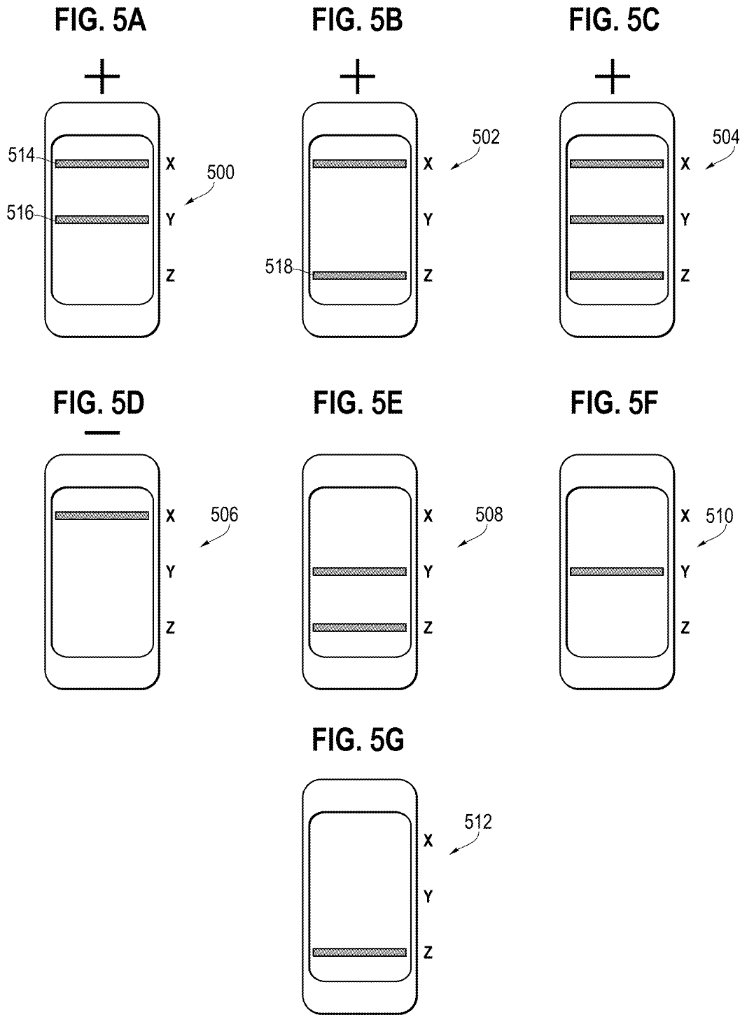

Referring to FIGS. 5A, 5B, 50, 5D, 5E, 5F, and 5G, examples of different respective possible biomarker diagnostic test results 500, 502, 504, 506, 508, 510, and 512 that may provide an indication of a level of AFP are illustrated. In an example, positive test results 500, 502, 504 may provide a visual indication of a level of AFP in a sample above a predetermined level. In an example, a device 100 may include a test cassette connected to needle 102, whereby a sample may feed through needle 102 from a puncture site and provide some of the sample to the test cassette. Buffer may then be added to the sample collected in the test cassette.

In an example, a test cassette may be made of plastic and may enclose a point-of-care membrane-based test strip. The test strip may require only a single drop of a sample. In certain examples, a test cassette may include a free-standing enzyme-modified responsive polymer membrane-based biosensor. In other examples, a test cassette may include a rapid antigen test. In still other examples, a test cassette may include alternate nucleic acid amplification methods. In still other examples, a test cassette may be connected to a smartphone or a tablet configured to receive results electronically by transmission of data from a test cassette.

The sample in the test cassette may be connected to a meter which transmits a current to the sample. Test results 500, 502, 504, 506, 508, 510, or 512 may then be read off of the test cassette after about 15 minutes. An example of a positive test result 500 may include a visual indication including a control indication 514 and a first indication 516. Another example of a positive test result 502 may include a visual indication including a control indication 514 and a second indication 518. Another example of a positive test result 504 may include a visual indication including a control indication 514, a first indication 516, and a second indication 518. In an example, a negative test result 506 may provide a visual indication of a level of AFP in a sample below a predetermined level, including only a control indication 514. Control indication 514 may confirm that the test was accurately performed. Invalid test results 508, 510, and 512 include only first indication 516 and/or second indication 518. The test cassettes may be used to take blood from the hepatic vein and the portal vein to indicate signs of the growth of AFP level in the blood, or can show decreasing signs through the liver.

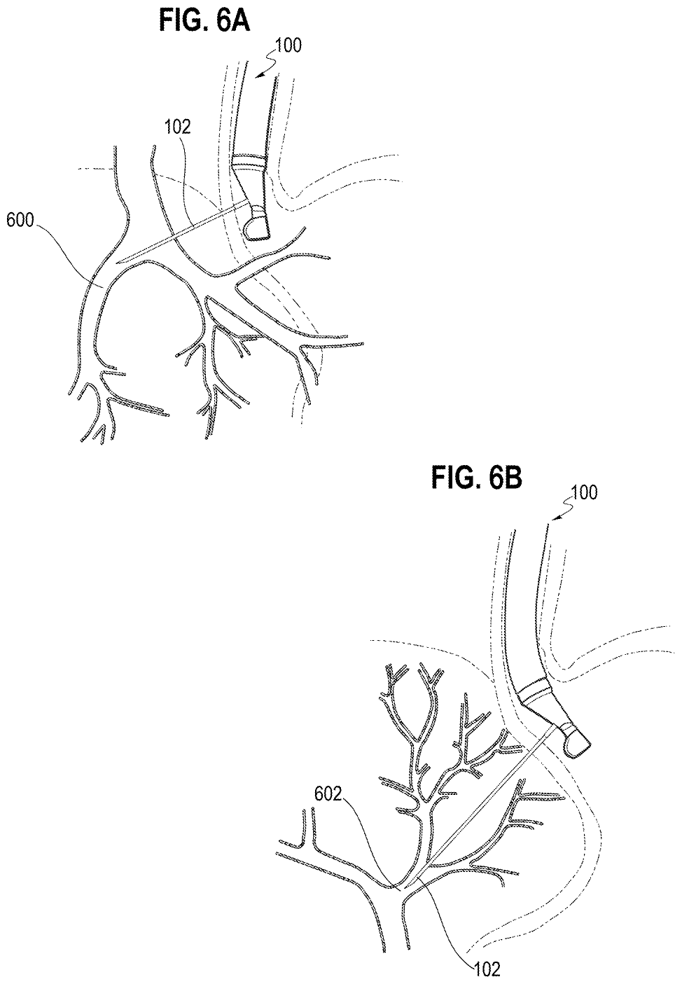

FIG. 6A illustrates an example of the insertion of needle 102 of device 100 inserted into a mammal's hepatic vein 600 to draw a sample for testing for one or more diagnostic biomarkers. FIG. 6B illustrates an example of the insertion of needle 102 of device 100 inserted into a mammal's portal vein 602 to draw a sample for testing for one or more diagnostic biomarkers.

In an example, the present disclosure provides a method of providing medical data about a mammal. The method includes: inserting a needle into a vein of the mammal; measuring the portal pressure in the vein; collecting a sample from the vein; measuring a diagnostic biomarker within the sample; and removing the needle from the vein; wherein an indication of a measurement of the diagnostic biomarker is provided prior to the removing.

In certain examples, the method may further include: analyzing the indication to provide information about a state of the health of the mammal. In other examples, the device may further include a digital microfluidic biochip. In still other examples, the diagnostic biomarker may further include: a white blood cell count, a red blood cell count, a platelet count, a hematocrit, one or more of the red blood cell indices, or combinations thereof. In still other examples, the diagnostic biomarker may include: apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, or combinations thereof. In still other examples, the diagnostic biomarker may include circulating tumor cells, exosomes, or combinations thereof. In still other examples, the device may include a test cassette connected to the needle. In still other examples, the diagnostic biomarker may include Alpha FetoProtein. In still other examples, the measuring the diagnostic biomarker may include: providing an amount of the sample from the needle to the test cassette; adding buffer to the sample in the test cassette; transmitting a current to the sample in the test cassette; and reading a test result on the test cassette. In still other examples, the vein may be the hepatic vein, the portal vein, or both. In still other examples, the device may further include a pressure transducer. In still other examples, the device may further include a sample measurement device attached to the needle near a distal tip of the needle, the sample measurement device configured to communicate an indication of a measurement to an external receiver. In still other examples, the diagnostic biomarker may be bilirubin. In still other examples, the mammal may be a human. In still other examples, the collecting a sample may include performing a biopsy.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

The subject-matter of the disclosure may also relate, among others, to the following aspects:

A first aspect relates to a medical device, comprising: a tubular body defining a lumen extending longitudinally therethrough, the tubular body defining a longitudinal axis, the tubular body comprising: a needle comprising the lumen extending longitudinally therethrough; a handle proximal to the needle; a stopcock proximal to the handle, the stopcock configured to close or open the lumen to flow of a sample proximal to the stopcock; a connecting tube proximal to the stopcock, the connecting tube comprising the lumen; a pressure transducer proximal to the connecting tube; and a syringe proximal to the pressure transducer; and a sample measurement device attached to the handle or to the needle near a distal tip of the needle; wherein the device is configured to provide a measurement of portal pressure in a vein of a mammal and an indication of a measurement of a diagnostic biomarker in a sample from the vein before the needle is removed from the vein.

A second aspect relates to the medical device of aspect 1, wherein the sample measurement device is configured to detect a bilirubin concentration.

A third aspect relates to the medical device of aspect 1 or 2, wherein the sample measurement device is a digital microfluidic biochip.

A fourth aspect relates to the medical device of aspects 1 to 3, wherein the diagnostic biomarker comprises: a white blood cell count, a red blood cell count, a platelet count, a hematocrit, one or more of the red blood cell indices, or combinations thereof.

A fifth aspect relates to the medical device of aspects 1 to 4, wherein the diagnostic biomarker comprises: apolipoprotein F, lipopolysaccharide-binding protein, ficolin-2, apolipoprotein D, kininogen-1, apolipoprotein M, thrombospondin-1, IgG F C-binding protein, cystatin-C, alpha 1-acid glycoprotein 2, leucine-rich alpha-2-glycoprotein, or combinations thereof.

A sixth aspect relates to the medical device of aspects 1 to 5, wherein the diagnostic biomarker comprises circulating tumor cells, exosomes, or combinations thereof.

A seventh aspect relates to the medical device of aspects 1 to 6, wherein the diagnostic biomarker comprises Alpha FetoProtein.

An eighth aspect relates to the medical device of aspects 1 to 7, wherein the vein is the hepatic vein, the portal vein, or both.

A ninth aspect relates to the medical device of aspects 1 to 8, wherein the sample measurement device is configured to communicate an indication of a measurement to an external receiver.

A tenth aspect relates to the medical device of aspects 1 to 9, further comprising a test cassette connected to the needle.

An eleventh aspect relates to a method of providing medical data about a mammal, comprising: inserting a needle into a vein of the mammal; measuring the portal pressure in the vein; collecting a sample from the vein; measuring a diagnostic biomarker within the sample; and removing the needle from the vein; wherein an indication of a measurement of the diagnostic biomarker is provided prior to the removing.

A twelfth aspect relates to the method of aspect 11, wherein the device further comprises a digital microfluidic biochip.

A thirteenth aspect relates to the method of aspect 11 or 12, wherein the diagnostic biomarker comprises: a white blood cell count, a red blood cell count, a platelet count, a hematocrit, one or more of the red blood cell indices, or combinations thereof.

A fourteenth aspect relates to the method of aspects 11 to 13, wherein the diagnostic biomarker comprises: apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, or combinations thereof.

A fifteenth aspect relates to the method of aspects 11 to 14, wherein the diagnostic biomarker comprises circulating tumor cells, exosomes, or combinations thereof.

A sixteenth aspect relates to the method of aspects 11 to 15, wherein the device comprises a test cassette connected to the needle.

A seventeenth aspect relates to the method of aspects 11 to 16, wherein the measuring the diagnostic biomarker comprises: providing an amount of the sample from the needle to the test cassette; adding buffer to the sample in the test cassette; transmitting a current to the sample in the test cassette; and reading a test result on the test cassette.

An eighteenth aspect relates to the method of aspects 11 to 17, wherein the vein is the hepatic vein, the portal vein, or both.

A nineteenth aspect relates to the method of aspects 11 to 18, wherein the mammal is a human.

In addition to the features mentioned in each of the independent aspects enumerated above, some examples may show, alone or in combination, the optional features mentioned in the dependent aspects and/or disclosed in the description above and shown in the figures.

We claim:

1. A medical device, comprising:
a tubular body defining a lumen extending longitudinally therethrough, the tubular body defining a longitudinal axis, the tubular body comprising:
a needle comprising the lumen extending longitudinally therethrough;
a handle proximal to the needle;
a stopcock proximal to the handle, the stopcock configured to close or open the lumen to flow of a sample proximal to the stopcock;
a connecting tube proximal to the stopcock, the connecting tube comprising the lumen;
a pressure transducer proximal to the connecting tube; and
a syringe proximal to the pressure transducer; and
a sample measurement device attached to the handle or to the needle near a distal tip of the needle;
wherein the device is configured to provide a measurement of portal pressure in a vein of a mammal and an indication of a measurement of a diagnostic biomarker in a sample from the vein before the needle is removed from the vein.

2. The device of claim 1, wherein the sample measurement device is configured to detect a bilirubin concentration.

3. The device of claim 1, wherein the sample measurement device is a digital microfluidic biochip.

4. The device of claim 1, wherein the diagnostic biomarker comprises: a white blood cell count, a red blood cell count, a platelet count, a hematocrit, one or more of red blood cell indices, or combinations thereof.

5. The device of claim 1, wherein the diagnostic biomarker comprises: apolipoprotein F, lipopolysaccharide-binding protein, ficolin-2, apolipoprotein D, kininogen-1, apolipoprotein M, thrombospondin-1, IgG F C-binding protein, cystatin-C, alpha 1-acid glycoprotein 2, leucine-rich alpha-2-glycoprotein, or combinations thereof.

6. The device of claim 1, wherein the diagnostic biomarker comprises circulating tumor cells, exosomes, or combinations thereof.

7. The device of claim 1, wherein the diagnostic biomarker comprises Alpha FetoProtein.

8. The device of claim 1, wherein the vein is a hepatic vein, a portal vein, or both.

9. The device of claim 1, wherein the sample measurement device is configured to communicate an indication of a measurement to an external receiver.

10. The device of claim 1, further comprising a test cassette connected to the needle.

11. A method of providing medical data about a mammal, comprising:
inserting a needle of a medical device into a vein of the mammal;
measuring a portal pressure in the vein;
collecting a sample from the vein;
measuring a diagnostic biomarker within the sample; and
removing the needle from the vein;
wherein an indication of a measurement of the diagnostic biomarker is provided prior to the removing.

12. The method of claim 11, wherein the medical device further comprises a digital microfluidic biochip.

13. The method of claim 11, wherein the diagnostic biomarker comprises: a white blood cell count, a red blood cell count, a platelet count, a hematocrit, one or more of red blood cell indices, or combinations thereof.

14. The method of claim 11, wherein the diagnostic biomarker comprises: apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, or combinations thereof.

15. The method of claim 11, wherein the diagnostic biomarker comprises circulating tumor cells, exosomes, or combinations thereof.

16. The method of claim 11, wherein the medical device comprises a test cassette connected to the needle.

17. The method of claim 11, wherein the measuring the diagnostic biomarker comprises:
providing an amount of the sample from the needle to a test cassette;
adding buffer to the sample in the test cassette;
transmitting a current to the sample in the test cassette; and
reading a test result on the test cassette.

18. The method of claim 11, wherein the vein is a hepatic vein, a portal vein, or both.

19. The method of claim 11, wherein the mammal is a human.

20. A method of providing medical data about a mammal, comprising:
inserting a needle of a device into a vein of the mammal, the device further comprising a digital microfluidic biochip;
measuring a portal pressure in the vein;
collecting a sample from the vein;
measuring a diagnostic biomarker within the sample; and
removing the device from the vein;
wherein an indication of a measurement of the diagnostic biomarker is provided prior to the removing; and
wherein the diagnostic biomarker comprises: a white blood cell count, a red blood cell count, a platelet count, a hematocrit, one or more of red blood cell indices, apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, circulating tumor cells, exosomes, or combinations thereof.
